(19) United States
(12) Patent Application Publication
(10) Pub. No.: US 2024/0117359 A1
(43) Pub. Date: Apr. 11, 2024

(54) RECOMBINANT CONSTRUCT FOR CANCER TREATMENT

(71) Applicant: COUNCIL OF SCINTIFIC AND INDUSTRIAL RESEARCH AN INDIAN REGISTERED BODY INCORPOR, Delhi (IN)

(72) Inventors: Shantanu CHOWDHURY, Delhi (IN); Subhajit DUTTA, Delhi (IN); Ananda Kishore MUKHERJEE, Delhi (IN); Antara SENGUPTA, Delhi (IN); Shuvra Shekhar ROY, Delhi (IN); Sulochana BAGRI, Delhi (IN)

(21) Appl. No.: 18/192,467

(22) Filed: Mar. 29, 2023

(30) Foreign Application Priority Data
Mar. 29, 2022 (IN) 202211019412

(51) Int. Cl. C12N 15/113; C12N 9/22

(52) U.S. Cl. CPC C12N 15/1138; C12N 9/22; C12N 2310/20; C12N 2310/3513; C12N 2320/32

(57) ABSTRACT

The present invention relates to novel targeted anti-cancer therapeutic approach. It is a plasmid-based delivery of dCas9 (dead Cas9) conjugated K293R TRF2 (telomeric repeat-binding factor2) along with a guide RNA for suppressing IL1R1 (interleukin 1 receptor type 1) activation in the tumour microenvironment. The present invention can be independently used in cancer therapy or can be used combined with other immune checkpoint therapy. Due to the use of dCas9 (dead Cas9) and sgRNA (short guide RNA), the present invention has limited or no off-target effect.

### CROSS-REFERENCE TO RELATED APPLICATION

This Non-provisional application claims priority under 35 U.S.C. § 119(a) to India Patent Application No. 202211019412, filed on 29 Mar. 2022, the entire contents of which is hereby incorporated by reference in its entirety.

### INCORPORATION BY REFERENCE OF SEQUENCE LISTING

A Sequence Listing is provided herewith as an xml file, “2339157.xml” created on May 31, 2023, and having a size of 38,080 bytes. The content of the xml file is incorporated by reference herein in its entirety.

### FIELD OF THE INVENTION

The present invention relates to a construct for cancer therapy. More particularly the invention relates to targeted suppression of IL1R1 (interleukin 1 receptor type 1) expression using dCas9 (dead Cas9) protein conjugated post translationally modified TRF2 (telomeric repeat-binding factor 2) via a sgRNA (small guide RNA) against IL1R1 (interleukin 1 receptor type 1) promoter. This complex of constructs acts as a negative regulator of IL1R1 (interleukin 1 receptor type 1) in the tumor microenvironment, which helps to reduce the burden of M2 type of TAM (tumor associated macrophage).

### BACKGROUND OF THE INVENTION

IL1 (interleukin 1) is one of the key proinflammatory cytokines modulating the immune architecture in tumour microenvironment. Among all the ligands, IL1β (interleukin 1 beta) and among all the receptors, IL1R1 (interleukin 1 receptor type 1) are mostly studied. IL1R1 (interleukin 1 receptor type 1) signalling cascade can initiate the NFκB (Nuclear factor kappa B) signalling, which further regulates IL1β (interleukin 1 beta) secretion². This feed forward loop induces bulk volume of IL1β (interleukin 1 beta) secretion in the tumour microenvironment. Interestingly IL1β (interleukin 1 beta) chemokine is a chemoattractant of various tumor associated macrophages, prevalently of M2 subtypes. Higher M2 recruitment leads to the impaired T cell function³. All these events lead to an immune suppressive tumor promoting condition. Owing to all these results, IL-1 (interleukin 1) has emerged as an important player in the cancer therapeutics⁴. Several clinical trials are ongoing, such as for IL-1 (interleukin 1) targeting agents, which include IL-1Ra (interleukin-1 receptor antagonist) (anakinra), the anti-IL-1b mAb (anti-interleukin 1 beta monoclonal antibody) (canakinumab), anti-IL-1a mAbs (anti-interleukin 1 alpha monoclonal antibody) (MABp1), and a fusion protein consisting of the ligand-binding regions of IL1R1 (interleukin 1 receptor type 1) and IL-IRAcP (interleukin-1 receptor accessory protein) linked to the Fc (fragment crystallizable) region of human IgGI (immunoglobulin G type1) (rilonacept), etc¹. Most of these are highly expensive, thermo labile and not easily available.

It was initially observed that TRF2 (telomeric repeat-binding factor 2), which is a shelterin protein component can regulate transcription also via occupying majorly the gene promoters and subsequently via recruiting histone modifiers⁵. It was previously known that TRF2 (telomeric repeat-binding factor 2) can interact with p300 for histone modification⁶. Very recently it was observed that TRF2 (telomeric repeat-binding factor 2) acts as an activator of IL1R1 (interleukin 1 receptor type 1) via recruiting p300⁷. Several domain mutants and post translational modifications of TRF2 (telomeric repeat-binding factor 2) were screened and then the IL1R1 (interleukin 1 receptor type 1) expression was measured. It was found that a particular PTM (post translational modification) K293R of TRF2 (telomeric repeat-binding factor 2) could hinder IL1R1 (interleukin 1 receptor type 1) activation. This finding has guided to delve deeper for targeted silencing of IL1R1 (interleukin 1 receptor type 1). It was found that using CRISPR based targeting strategy can deliver the protein to particular locus, i.e., IL1R1 (interleukin 1 receptor type 1) promoter. So, dead Cas9 was used, which is mutated in such a way that it lacks endonuclease activity. The K293R TRF2 (telomeric repeat-binding factor 2) was fused with dead Cas9 in the same frame. Guiding the fused protein to the locus leads to silencing of IL1R1 (interleukin 1 receptor type 1) in gene level as well as in protein level. For further validation, wild type TRF2 (telomeric repeat-binding factor 2) was fused with dCas9 (dead Cas9) in same manner and delivered with guide RNA, which resulted in higher IL1R1 (interleukin 1 receptor type 1) expression. Further testing was carried out in MDAMB231 (breast cancer) and s HT1080 (fibrosarcoma) cell lines.

### OBJECTIVE OF THE INVENTION

The main objective of the present invention is to provide a targeted molecular biological approach to precisely suppress IL1R1 (interleukin 1 receptor type 1) expression in cancer cells in the tumour microenvironment. IL1R1 (interleukin 1 receptor type 1) is a receptor of IL1β (interleukin 1 beta), one of the major chemoattractant of M2 type TAM (tumor associated macrophage), which subsequently suppresses anti-tumour adaptive immunity (via T cells). This breakthrough can be used synergically with immune checkpoint inhibitors for better therapeutic potential and lower cost.

Another objective is to standardize the dosage and delivery of the complex in the microenvironment to test the efficacy of the invention on cellular toxicity, cell proliferation, induction of senescence and outcome compared to conventional therapeutic practices.

### SUMMARY OF THE INVENTION

Accordingly, the invention provides two plasmids to impede IL1R1 (interleukin 1 receptor type 1) expression. One of the plasmids encodes a deadCas9 conjugated PTM (post translational modification) (K293R) of TRF2 (telomeric repeat-binding factor 2). The other plasmid expresses a guide RNA designed against IL1R1 (interleukin 1 receptor type 1) promoter. Combination of these two will inhibit the wild type TRF2 (telomeric repeat-binding factor 2) binding to the promoter region, which can activate IL1R1 (interleukin 1 receptor type 1) in a feed forward loop manner. The plasmids can be delivered in cell culture using commercially available transfection reagents.

The present invention sheds light on a heretofore unknown molecular mechanism of IL1R1 (interleukin 1 receptor type 1) signalling in the tumor microenvironment. The signalling is critical of M2 infiltration in the microenvironment which can potentially arrest the anti-tumor immunity. The proposed therapy can reduce the M2 infiltration and enhance the chances of significantly better therapeutic responses.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The invention may be better understood by reference to the drawings in combination with the detailed description of the specific embodiments presented herein.

FIG. 1 depicts dCas9 K293R_TRF2 & WT_TRF2 expression in MDAMB231 cells and their role in IL1R1 expression, in accordance with an embodiment of the present disclosure.

FIG. 2 depicts dCas9 K293R_TRF2 & WT_TRF2 expression in HT1080 cells and their role in IL1R1 expression, in accordance with an embodiment of the present disclosure.

FIG. 3 depicts dCas9 K293R_TRF2 & WT_TRF2 expression in MDAMB231 cells and their role in NFκB signalling, in accordance with an embodiment of the present disclosure.

FIGS. 4A and 4B depict (A) dCas9 K293R_TRF2 & WT_TRF2 expression in MDAMB231 & HT1080 cells and their role in IL1R1 mRNA expression (B) dCas9 K293R_TRF2 & WT_TRF2 expression in MDAMB231 & HT1080 cells and their off-target effect check, in accordance with an embodiment of the present disclosure.

FIG. 5 depicts Control conditions of dCas9 K293R TRF2—IL1R1 sgRNA experiment, in accordance with an embodiment of the present disclosure.

FIGS. 6A and 6B depict (A) Plasmid map of dCas9_TRF2_K293R (SEQ ID 1); (B) Plasmid map of sgRNA vector targeting IL1R1, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

Sequences used in the present disclosure:

SEQ ID 1: Plasmid Sequence of dCas9_TRF2_K293R

SEQ ID 2: Plasmid Sequence of IL1R1 guide RNA

SEQ ID 3: sgRNA sequence present in SEQ ID 2

SEQ ID 4: Functional Protein Sequence of dCas9_TRF2_K293R

SEQ ID 5: Cloning Primer TRF2 in dCAS9-VP64 containing BamHI restriction enzyme site

SEQ ID 6: Cloning Primer TRF2 in dCAS9-VP64 containing NheI restriction enzyme site

SEQ ID 7: Cloning Primer mCherry in PX333 containing AgeI restriction enzyme site

SEQ ID 8: Cloning Primer mCherry in PX333 containing AgeI restriction enzyme site

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

In an embodiment of the present disclosure, there is provided a CRISPR-Cas based gene-editing system of a target gene comprising vector encoding: a) post-translationally modified TRF2 (telomeric repeat-binding factor 2) operably linked to a nucleotide sequence encoding a RNA-directed dead Cas9 (dCas9) having SEQ ID 1; b) a promoter operably linked to a nucleotide sequence encoding a CRISPR-Cas9 system gRNA (guide RNA) having SEQ ID 2, wherein the gRNA (guide RNA) targets and hybridizes with the target sequence and directs the RNA-directed dead Cas9 to the DNA locus, wherein, components a) and b) are located on different vectors.

In another embodiment of the present disclosure, the RNA-directed nuclease is a dCas9 protein.

In another embodiment of the present disclosure, the gRNA (guide RNA) sequence (SEQ ID 3) is targeting IL1R1.

In an embodiment of the present disclosure, there is provided a nucleic acid construct as disclosed herein comprises nucleic acid sequence encoding a dCas9 (dead Cas9) fusion protein having SEQ ID NO 1.

In an embodiment of the present disclosure, there is provided a nucleic acid construct as claimed in claim 1b) comprising a guide RNA having SEQ ID NO 2.

In an embodiment of the present disclosure, there is provided a method of preparing the construct as disclosed herein comprising the steps of: a. providing TRF2 (telomeric repeats binding factor 2); b. mutating TRF2 obtained in step (a) by site directed mutagenesis on the lysine 293 residue; and c. cloning the sequence obtained in step (b) into the dCas9-VP64_GFP backbone.

In an embodiment of the present disclosure, there is provided a method of preparing the construct as disclosed herein comprising the steps of: a. providing pX333 plasmid; b. providing mCherry reporter gene; c. synthesizing sgRNA having SEQ ID No 3; d. cloning mCherry reporter gene obtained in step (b) in pX333 plasmid obtained in step (a) to obtain mCherry pX333plasmid; and e. cloning sgRNA into the plasmid obtained in step (d).

In another embodiment of the present disclosure, the dCas9 (dead Cas9) fusion protein comprises dCas9 (dead Cas9) fused with a post-translationally modified TRF2 (telomeric repeat-binding factor 2) acting as a transcriptional repressor of the target gene IL1R1 (interleukin 1 receptor type 1).

In an embodiment of the present disclosure, there is provided a formulation comprising the construct as disclosed herein along with 10 mM Tris-Cl buffer and mixed in 1:1 ratio with the delivery agent.

In an embodiment of the present disclosure, there is provided a formulation as disclosed herein, wherein the CRISPR-dCas based gene-editing system is used for targeting other genes including, but not limiting to, IL1R1 (interleukin 1 receptor type 1).

In an embodiment of the present disclosure, there is provided a method for selectively targeting regulation of a single gene in cancer comprising the steps. a) providing construct having SEQ ID no 1 and 2; b) providing 10 mM Tris-Cl buffer; c) mixing the constructs in the ratio of 1:1 at pH 8.5. and d) targeting IL1R1 (interleukin 1 receptor type 1) along with a delivery agent.

In an embodiment of the present disclosure, there is provided a targeted anti-cancer therapeutic method modulating the expression of a target nucleic acid sequence in a cell comprising: introducing into the cell a nucleic acid sequence encoding a dCas9 (deadCas9) fusion protein and a guide RNA, wherein the dCas9 (dead Cas9) fusion protein comprises dCas9 (dead Cas9) fused with a post-translationally modified TRF2 (telomeric repeat-binding factor 2), wherein the Cas9 fusion protein and the guide RNA are expressed and co-localize at a target site and modulate the expression of the target gene.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Example 1

### 1. Construction of the Plasmids

This invention involves two plasmids to impede IL1R1 expression. One of the plasmid encodes a dCas9 (dead Cas9) conjugated PTM (post translational modification) K293R of TRF2 (telomere repeats-binding factor 2). The other one expresses a gRNA (guide RNA) designed against IL1R1 (interleukin 1 receptor type 1) promoter. Combination of these two inhibits the wild type TRF2 (telomere repeats-binding factor 2) binding to the promoter region which activates IL1R1 (interleukin 1 receptor type 1) in a feed forward loop manner. The plasmid was delivered in cell culture using commercially available transfection reagents.

The two plasmids were prepared as follows:

A catalytically inactive SpCas9 (dCas9) N-terminally was fused with full length Telomere Repeat binding Factor 2 (TRF2) protein.

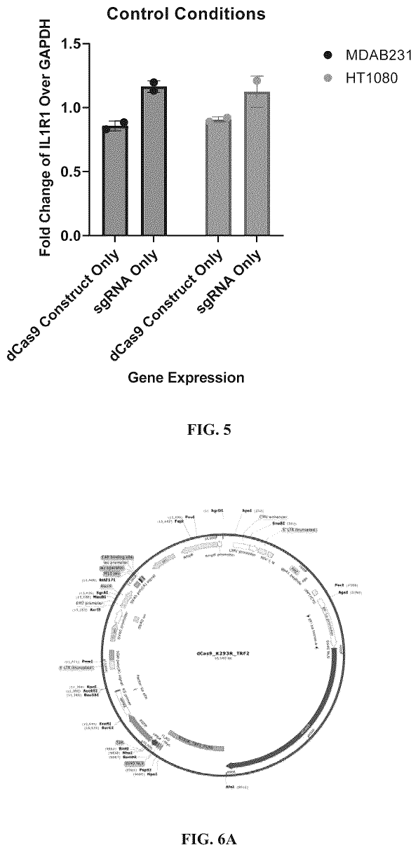

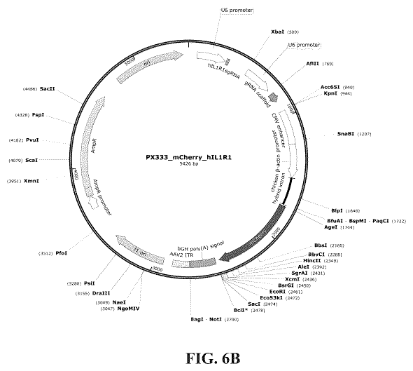

a) To generate the plasmid (FIG. 6A), CDS (coding DNA sequence) of TRF2 (telomere repeats-binding factor 2) was first cloned into Addgene plasmid #61422 (in place of VP64 protein) from the existing TRF2 (telomere repeats-binding factor 2) overexpression pCMV6 backbone plasmid. A 1600 bp amplicon was generated following below reaction conditions (Table 1) and thermal cycling (Table 2) using Thermo Scientific Phusion High-Fidelity DNA Polymerase.

TABLE 1

| | Volume (Total |
|---|---|
| Component | 20 μl) |
| 5X GC buffer | 4 |
| 10 μM Forward Primer ^{#} | 0.5 |
| 10 μM Reverse Primer ^{#} | 0.5 |
| 10 ng Template (pCMV6 TRF2 plasmid) | 2 |
| 10 mM dNTP mix | 0.4 |
| DMSO | 0.6 |
| 50 mM MgCl2 | 0.2 |
| Nuclease Free water | 11.7 |
| Phusion DNA Polymerase | 0.1 |

TABLE 2

| | Temperature | | | |
|---|---|---|---|---|
| Cycle Step | (° C.) | Time | | Cycles |
| Initial | 98 | 30 | s | 1x |
| Denaturation | | | | |
| Denaturation | 98 | 10 | s | |
| Annealing | 70 * | 30 | s | |
| Extension | 72 | 50 | s ** | 35x |
| Final extension | 72 | 10 | min | |

| | | |
|---|---|---|
| 4 | hold | 1x |

In Table 2, “*” denotes Temperature selected post gradient PCR at (55, 58, 60, 64, 78, 70)° C.

In Table “**” denotes considering 30-40 s/kb extension rate

The sequence of the forward primer for FWD C-term TRF2BamHI is as follows:

(SEQ ID NO: 5)
5′ AATGGATCCATGGCGGGAGGAGGCGGGAGTAGCGA

The sequence of the reverse primer for REV C-term TRF2NheI is as follows:

(SEQ ID NO: 6)
5′ AATGCTAGCAACCTTATCGTCGTCATCCTTGTAAT
CCAGGATATCATTTGCTG

b) Full length TRF2 (telomere repeats-binding factor 2) with 293^{rd} residue mutation K to R was also similarly amplified from appropriate pCMV6 TRF2 (telomere repeats-binding factor 2) K293R (lysine 293 modified to arginine) overexpression plasmid following above mentioned protocol.

c) Amplicons and backbone dCas9-VP64_GFP (addgene 61422) plasmid was restriction digested with BamHI (from New England Biolabs) and NheI (from New England Biolabs) enzymes. The plasmid backbone was further incubated in Shrimp alkaline phosphatase treatment to reduce self-ligation events in subsequent ligation reaction.

d) Overnight ligation at 16° C. was setup at 1:7 and 1:5 (vector: amplicon) ratio and then transformed in DH5a chemical competent cells for colony formation.

e) Colonies were then screened by colony-PCR (using the same above-mentioned cycle conditions) and restriction digestion confirmatory checks.

A guide RNA, for IL1R1 (interleukin 1 receptor type 1) promoter expressing plasmid (FIG. 6B) with mCherry expression for visual marking was prepared by genetically engineering the Addgene plasmid #64073 pX333 to substitute the Cas9 with mCherry sequence.

For mCherry substitution into pX333 plasmid, mCherry CDS (coding DNA sequence) was amplified from pDECKO-mCherry plasmid following the below conditions listed in Table 3 and Table 4.

TABLE 3

| | Volume (Total |
|---|---|
| Component | 20 μl) |
| 5X GC buffer | 4 |
| 10 μM Forward Primer ^{#} | 0.5 |
| 10 μM Reverse Primer ^{#} | 0.5 |
| 10 ng Template (pDECKO-mCherry | 2 |
| plasmid) | |
| 10 mM dNTP mix | 0.4 |
| DMSO | 0.6 |
| 50 mM MgCl2 | 0.2 |
| Nuclease Free water | 11.7 |
| Phusion DNA Polymerase | 0.1 |

TABLE 4

| | Temperature | | | |
|---|---|---|---|---|
| Cycle Step | (° C.) | Time | | Cycles |
| Initial | 98 | 30 | s | 1x |
| Denaturation | | | | |
| Denaturation | 98 | 10 | s | |
| Annealing | 70 * | 30 | s | |
| Extension | 72 | 25 | s ** | 30x |
| Final extension | 72 | 10 | min | |

| | | |
|---|---|---|
| 4 | hold | 1x |

In Table 4 “*” denotes the temperature selected post gradient PCR at (55, 58, 60, 64, 78, 70)° C.

In Table 4 “**” denotes considering 30-40 s/kb extension rate

In Table 3 “#” denotes Cloning of mCherry into pX333 plasmid by removing Cas9 (ACCGGT—AgeI GAATTC—EcoRI)

The sequence of the mCherry forward primer is as follows.

(SEQ ID NO: 7)
5′ TATAACCGGTCGCCACCATGGTGAGCAAG

The sequence of the mCherry reverse primer is as follows.

(SEQ ID NO: 8)
5′ TATAGAATTCTTACTTGTACAGCTCGTCCATGCCGCC

a) Amplicons and pX333 plasmid were restriction digested with AgeI (from New England Biolabs) and EcoRI (from New England Biolabs) enzymes. The plasmid backbone was further incubated in Shrimp alkaline phosphatase treatment to reduce self-ligation events in subsequent ligation reaction.

b) Overnight ligation at 16° C. was setup at 1:7 and 1:5 (vector: amplicon) ratio and then transformed in DH5a chemical competent cells for colony formation.

Colonies were then screened by colony-PCR (above mentioned cycle conditions) and checked with restriction digestion for further confirmation.

2.1. Then the sgRNA scaffold plasmid with mCherry was used to clone the 20 bp long gRNA (guide RNA) for IL1R1 (interleukin 1 receptor type 1) promoter

(SEQ ID NO: 3)
(GCTGCCAATGGGTGGAGTCTT).

a) sgRNA (short guide RNA) oligos were annealed following the below protocol listed in Table 5 and table 6.

TABLE 5

| | Volume (Total |
|---|---|
| Component | 10 μl) |
| T4 DNA ligation buffer | 1 |
| 100 μM Forward Primer | 1 |
| 100 μM Reverse Primer | 1 |
| Nuclease Free water | 6 |
| T4 Polynucleotide Kinase | 1 |
| (PNK) | |

TABLE 6

| Cycle Step | Temperature (° C.) | Time | | Cycles |
|---|---|---|---|---|
| Incubation | 37 | 30 | min | 1x |
| Denaturation | 95 | 5 | min | 1x |
| Slow cooling | 95 ramp down to | 5° | C./min | 1x |

25

b) Plasmid cloned in 2.1. was then digested with BbsI enzyme and overnight ligation at 16° C. reaction was set up with annealed oligos following below protocol listed in Table 7.

TABLE 7

| Component | Volume (Total 10 μl) |
|---|---|
| T4 DNA ligation buffer | 1 |
| 20 ng BbsI digested vector | 2 |
| 1/200 th diluted annealed oligo of | 1 |
| 2.2.a) | |
| Nuclease Free water | 5 |
| T4 DNA ligase | 1 |

c) Ligation product was then transformed in DH5a, chemically competent cells for colony formation.

d) Colonies were then screened by colony-PCR using hU6 Forward primer and sgRNA reverse oligo primers using reaction conditions and cycling conditions as listed in Table 8 and Table 9, respectively.

TABLE 8

| | Volume (Total |
|---|---|
| Component | 25 μl) |
| 10X Std. Taq buffer | 2.5 |
| 10 μM Forward Primer | 0.5 |
| 10 μM Reverse Primer | 0.5 |
| 10 ng Template | 1 |
| (plasmid isolated from ligation colony) | |
| 10 mM dNTP mix | 0.5 |
| 25 mM MgCl2 | 0.4 |
| Nuclease Free water | 20.5 |
| Taq DNA Polymerase | 0.125 |

TABLE 9

| | Temperature | | | |
|---|---|---|---|---|
| Cycle Step | (° C.) | Time | | Cycles |
| Initial | 95 | 5 | min | 1x |
| Denaturation | | | | |
| Denaturation | 95 | 30 | s | |
| Annealing | 55 | 30 | s | |
| Extension | 72 | 30 | s* | 30x |
| Final extension | 72 | 5 | min | |

| | | |
|---|---|---|
| 4 | hold | 1x |

In table 9 “*” denotes 450 bp product was obtained upon successful cloning of sgRNA into the plasmid.

The plasmids were isolated by midi-prep method using Qiagen columns. The plasmids were eluted in 10 mM Tris-Cl, pH 8.5. The whole fusion protein sequence (SEQ ID NO: 4) is provided herein; in which the italics part is of dCas9 (dead Cas9), the underlined part is the actual effector TRF2 (telomeric repeat-binding factor 2), which has a modification at 293rd position marked in bold and the terminal bold part of the sequence is FLAG tag. The sequence is in a single letter amino acid code format.

DKKYSIGLAIGINSVGWAVITDEYKVPSKKFKVLGNTDRH
SIKKNLIGALLFDSGETAEATRLKRTARRRYTRRKNRICY
LQEIFSNEMAKVDDSFFHRLEESELVEEDKKHERHPIFGN
IVDEVAYHEKYPTIYHLRKKLVDSTDKADLRLIYLALAHM
IKFRGHFLIEGDLNPDNSDVDKLFIQLVQTYNQLFEENPI
NASGVDAKAILSARLSKSRRLENLIAQLPGEKKNGLFGNL
IALSLGLTPNFKSNEDLAEDAKLQLSKDTYDDDLDNLLAQ
IGDQYADLFLAAKNLSDAILLSDILRVNTEITKAPLSASM
IKRYDEHHQDLTLLKALVRQQLPEKYKEIFFDQSKNGYAG
YIDGGASQEEFYKFIKPILEKMDGTEELLVKLNREDLLRK
QRTFDNGSIPHQIHLGELHAILRRQEDFYPFLKDNREKIE
KILTFRIPYYVGPLARGNSRFAWMTRKSEETITPWNFEEV
VDKGASAQSFIERMTNFDKNLPNEKVLPKHSLLYEYFTVY
NELTKVKYVTEGMRKPAFLSGEQKKAIVDLLFKTNRKVTV
KQLKEDYFKKIECFDSVEISGVEDRENASLGTYHDLLKII
KDKDFLDNEENEDILEDIVLTLTLFEDREMIEERLKTYAH
LFDDKVMKQLKRRRYTGWGRLSRKLINGIRDKQSGKTILD
FLKSDGFANRNFMQLIHDDSLTFKEDIQKAQVSGQGDSLH
EHIANLAGSPAIKKGILQTVKVVDELVKVMGRHKPENIVI
EMARENQTTQKGQKNSRERMKRIEEGIKELGSQILKEHPV
ENTQLQNEKLYLYYLQNGRDMYVDQELDINRLSDYDVDAI
VPQSFLKDDSIDNKVLTRSDKNRGKSDNVPSEEVVKKMKN
YWRQLLNAKLITQRKFDNLTKAERGGLSELDKAGFIKRQL
VETRQITKHVAQILDSRMNTKYDENDKLIREVKVITLKSK
LVSDFRKDFQFYKVREINNYHHAHDAYLNAVVGTALIKKY
PKLESEFVYGDYKVYDVRKMIAKSEQEIGKATAKYFFYSN
IMNFFKTEITLANGEIRKRPLIETNGETGEIVWDKGRDFA
TVRKVLSMPQVNIVKKTEVQTGGFSKESILPKRNSDKLIA
RKKDWDPKKYGGFDSPTVAYSVLVVAKVEKGKSKKLKSVK
ELLGITIMERSSFEKNPIDFLEAKGYKEVKKDLIIKLPKY
SLFELENGRKRMLASAGELQKGNELALPSKYVNFLYLASH
YEKLKGSPEDNEQKQLFVEQHKHYLDEIIEQISEFSKRVI
LADANLDKVLSAYNKHRDKPIREQAENIIHLFTLTNLGAP
AAFKYFDTTIDRKRYTSTKEVLDATLIHQSITGLYETRID
LSQLGGDSAGGGGSGGGGSMAGGGGSSDGSGRAAGRRASR
SSGRARRGRHEPGLGGPAERGAGEARLEEAVNRWVLKFYF
HEALRAFRGSRYGDFRQIRDIMQALLVRPLGKEHTVSRLL
RVMQCLSRIEEGENLDCSFDMEAELTPLESAINVLEMIKT
EFTLTEAVVESSRKLVKEAAVIICIKNKEFEKASKILKKH
MSKDPTTQKLRNDLLNIIREKNLAHPVIQNFSYETFQQKM
LRFLESHLDDAEPYLLTMAKKALKSESAASSTGKEDKQPA
PGPVEKPPREPARQLRNPPTTIGMMTLKAAFRTLSGAQDS
EAAFAKLDQKDLVLPTQALPASPALKNKRPRKDENESSAP
ADGEGGSELQPKNKRMTISRLVLEEDSQSTEPSAGLNSSQ
EAASAPPSKPTVLNQPLPGEKNPKVPKGKWNSSNGVEEKE
TWVEEDELFQVQAAPDEDSTTNITKKQKWTVEESEWVKAG
VQKYGEGNWAAISKNYPFVNRTAVMIKDRWRTMKRLGMNT
RTRPLEQKLISEEDLAANDILDYKDDDDKV

### Example 2

### 11. Transfection of Encapsulated Plasmids: In Cellulo Model

One microgram of dCas9 (dead Cas9) WT_TRF2 (wild type telomeric repeats-binding factor 2) or K293R_TRF2 (lysine 293 modified to arginine telomeric repeats-binding factor 2) plasmids were mixed with 3 μl of transfection reagent (FuGene HD from Promega) and 200 μl of cell culture media (DMEM-high glucose or MEM); and then kept in room temperature for 10 minutes. This step helps in the encapsulation of the plasmid within the transfection reagent, which is a lipid rich formulation. Then the solution was poured dropwise on cells and incubated for 8 hours. Post that media was changed with normal cell culture media (without the transfection reagent). Within 24 hours EGFP (enhanced green fluorescence protein) signal was observed from cells, which indicated the expression of dCas9 conjugated TRF2 (telomeric repeats-binding factor 2). The transfected plasmids were maintained within the cells for 72 hours. To target the IL1R1 (interleukin 1 receptor type 1) promoter with the dCas9 (dead Cas9), gRNA (guide RNA) plasmid was transfected in same manner. Post gRNA (guide RNA) transfection, within another 24 hours mCherry fluorescence expression was observed under microscope. Cells were harvested at this point.

### Example 3

### III. Determination of the Inhibition Potential of the Invention: In Cellulo Model

RNA was isolated from the harvested cells with Trizol (Sigma) method and protein was isolated with RIPA (Sigma) buffer. cDNA (complementary DNA) was synthesized from the RNA. Post quantification IL1R1 (interleukin 1 receptor type 1) mRNA (messenger RNA) expression was measured using qRT PCR (quantitative real time polymerase chain reaction). To ensure the targeting efficiency, other TRF2 (telomeric repeats-binding factor 2) dependent genes (p21 for which TRF2 (telomeric repeats-binding factor 2) acts as a repressor⁸ and PDGFR (PDGFRB platelet derived growth factor receptor) for which TRF2 (telomeric repeats-binding factor 2) acts as an activator⁹) expression was also measured. The house-keeping gene GAPDH was used for normalization. Proteins were quantified using BCA (Bicinchoninic acid assay) method. Equal amount of protein was loaded on SDS PAGE (sodium dodecyl sulphate-polyacrylamide gel electrophoresis) for western blotting. IL1R1 (interleukin 1 receptor type 1), phospho NFkB (nuclear factor-κB) and total NFkB (nuclear factor-κB) expression was probed with primary antibody followed by complimentary secondary antibody. Then the blots were developed using HRP (horseradish peroxidase) chemiluminescent substrate from BioRad.

### Results

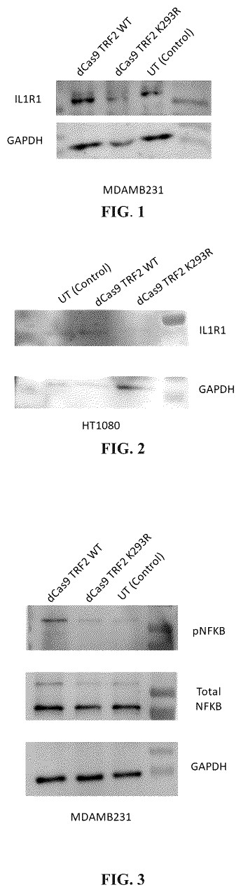

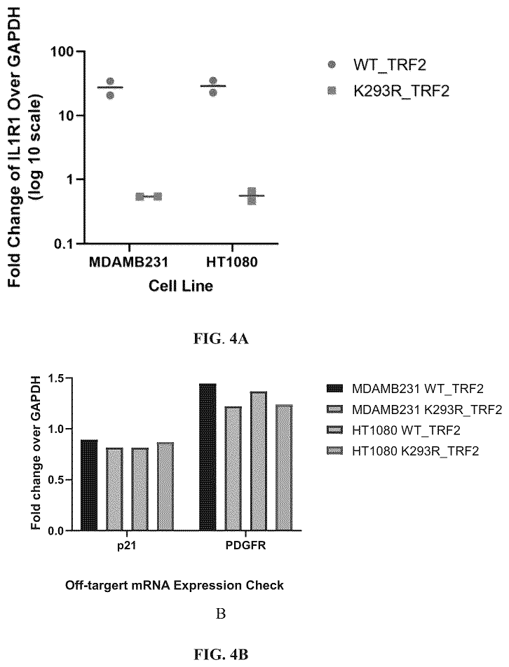

dCas9 K293R TRF2 and dCas9 WT TRF2 were expressed in MDAMB231 cell line. It was followed by guide RNA transfection. WT-TRF2 resulted in higher IL1R1 expression, whereas K293R-TRF2 resulted in lower IL1R1 expression (FIG. 1). Similarly, dCas9 K293R TRF2 and dCas9 WT TRF2 were expressed in HT1080 cell line. It was followed by guide RNA transfection. WT-TRF2 induced IL1R1 expression, whereas K293R-TRF2 inhibited IL1R1 expression (FIG. 2). dCas9 K293R TRF2 and dCas9 WT TRF2 expression in MDAMB231 and HT1080 cells resulted in differential IL1R1 mRNA expression as shown in FIG. 4A.

Further, dCas9 K293R TRF2 and dCas9 WT TRF2 expression in MDAMB231 resulted in altered NFkB signalling. WT TRF2 mediated higher IL1R1 led to phosphorylated NFkB (activation of NFkB signalling pathway) compared to the K293R TRF2 counterpart (FIG. 3).

It was also confirmed that dCas9 K293R TRF2 did not result in any off-target effects based on unaltered mRNA expression of p21 and PDGF, when compared to dCas9 WT TRF2 (FIG. 4B).

Finally, as shown in FIG. 5, it was confirmed that the combination of dCas9 K293R TRF2 and IL1R1 sgRNA constructs at a ratio of 1:1 is essential to functionally repress the IL1R1 expression.

### Advantages of the Invention

Immune therapy in recent days has drawn severe attention due to its promising results and less side effects compared to the traditional chemotherapy. But, in many cancers types the therapy shows mixed results due to less availability of killer T cells in the tumor microenvironment. In that premise, the present invention can be a game changer, which can inhibit M2 type of TAM (tumour associated macrophages) infiltration and subsequently increase the chances of killer T cell invasion. The PTM (post translational modification) is also a natural variant of TRF2 (telomeric repeats-binding factor 2) and the plasmids are very easy as well as cheap to grow and easy to store. With an aim in in vivo delivery the present invention cancels off-target effect, minimizes the cost and increases the chance of better outcome.

### REFERENCES

1. Mantovani, A., Dinarello, C. A., Molgora, M. & Garlanda, C. Interleukin-1 and Related Cytokines in the Regulation of Inflammation and Immunity. Immunity 50, 778-795 (2019).
2. Ji, Z., He, L., Regev, A. & Struhl, K. Inflammatory regulatory network mediated by the joint action of NF-kB, STAT3, and AP-1 factors is involved in many human cancers. Proc. Natl. Acad. Sci. 116, 9453-9462 (2019).
3. Garlanda, C. & Mantovani, A. Interleukin-1 in tumor progression, therapy, and prevention. Cancer Cell 39, 1023-1027 (2021).
4. Apte, R. N. et al. The involvement of IL-1 in tumorigenesis, tumor invasiveness, metastasis and tumor-host interactions. Cancer Metastasis Rev. 25, 387-408 (2006).

We claim:

1. A CRISPR-Cas based gene-editing system of a target gene comprising vector encoding:
a) post-translationally modified TRF2 (telomeric repeat-binding factor 2) operably linked to a nucleotide sequence encoding a RNA-directed dead Cas9 (dCas9) having SEQ ID 1;
b) a promoter operably linked to a nucleotide sequence encoding a CRISPR-Cas9 system gRNA (guide RNA) having SEQ ID 2, wherein the gRNA (guide RNA) targets and hybridizes with the target sequence and directs the RNA-directed dead Cas9 to the DNA locus;
wherein, components a) and b) are located on different vectors.

2. The CRISPR-Cas system as claimed in claim 1, wherein the RNA-directed nuclease is a dCas9 protein.

3. The CRISPR-Cas system as claimed in claim 1, wherein the gRNA (guide RNA) sequence (SEQ ID 3) is targeting IL1R1.

4. A nucleic acid construct as claimed in claim 1 comprises nucleic acid sequence encoding a dCas9 (dead Cas9) fusion protein having SEQ ID NO 1.

5. A nucleic acid construct as claimed in claim 1 comprising a guide RNA having SEQ ID NO 2.

6. A method of preparing the construct as claimed in claim 4 comprising the steps of:
a. providing TRF2 (telomeric repeats binding factor 2);
b. mutating TRF2 obtained in step (a) by site directed mutagenesis on the lysine 293 residue; and
c. cloning the sequence obtained in step (b) into the dCas9-VP64_GFP backbone.

7. A method of preparing the construct as claimed in claim 5 comprising the steps of:
a. providing pX333 plasmid;
b. providing mCherry reporter gene:
c. synthesizing sgRNA having SEQ ID No 3;
d. cloning mCherry reporter gene obtained in step (b) in pX333 plasmid obtained in step (a) to obtain mCherry pX333plasmid; and
e. cloning sgRNA into the plasmid obtained in step (d).

8. The nucleic acid construct as claimed in claim 4, wherein the dCas9 (dead Cas9) fusion protein comprises dCas9 (dead Cas9) fused with a post-translationally modified TRF2 (telomeric repeat-binding factor 2) acting as a transcriptional repressor of the target gene IL1R1 (interleukin 1 receptor type 1).

9. A formulation comprising the construct as claimed in claim 1 along with 10 mM Tris-Cl buffer and mixed in 1:1 ratio with the delivery agent.

10. A formulation as claimed in claim 9, wherein the CRISPR-dCas based gene-editing system is used for targeting other genes including, but not limiting to, IL1R1 (interleukin 1 receptor type 1).

11. A method for selectively targeting regulation of a single gene in cancer comprising the steps:
a) providing construct having SEQ ID no 1 and 2:
b) providing 10 mM Tris-Cl buffer;
c) mixing the constructs in the ratio of 1:1 at pH 8.5; and
d) targeting IL1R1 (interleukin 1 receptor type 1) along with a delivery agent.

12. A targeted anti-cancer therapeutic method modulating the expression of a target nucleic acid sequence in a cell comprising:
introducing into the cell a nucleic acid sequence encoding a dCas9 (deadCas9) fusion protein and a guide RNA,
wherein the dCas9 (dead Cas9) fusion protein comprises dCas9 (dead Cas9) fused with a post-translationally modified TRF2 (telomeric repeat-binding factor 2), wherein the Cas9 fusion protein and the guide RNA are expressed and co-localize at a target site and modulate the expression of the target gene.


SEQUENCE LISTING (8 sequences)

SEQ ID NO: 1 - DNA, 16140 bp, synthetic construct
  misc_feature 1..16140: note = Plasmid Sequence of dCas9_TRF2_K293R
  misc_feature 8046..9635: note = Functional_Sequence_dCas9_293 Arginine
  source 1..16140: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgagccccaagaagaagagaaaggtggaggccagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcatggcgggaggaggcgggagtagcgacggcagcgggcgggcagctggcaggcgggcgtcccgcagtagcgggcgggcccggcgggggcgccacgagccggggctggggggcccggcggagcgcggcgcgggggaggcacggctggaagaggcagtcaatcgctgggtgctcaagttctacttccacgaggcgctgcgggcctttcggggtagccggtacggggacttcagacagatccgggacatcatgcaggctttgcttgtcaggcccttggggaaggagcacaccgtgtcccgattgctgcgggttatgcagtgtctgtcgcggattgaagaaggggaaaatttagactgttcctttgatatggaggctgagctcacaccactggaatcagctatcaatgtgctggagatgattaaaacggaatttacactgacagaagcagtggtcgaatccagtagaaaactggtcaaggaagctgctgtcattatttgtatcaaaaacaaagaatttgaaaaggcttcaaaaattttgaaaaaacatatgtccaaggaccccacaactcagaagctgagaaatgatctcctgaatattattcgagaaaagaacttggcccatcctgttatccagaacttttcatatgagaccttccagcagaagatgctgcgcttcctggagagccacctggatgacgccgagccctacctcctcacgatggccaaaaaggctttgaaatctgagtccgctgcctcaagtacagggaaggaagataaacagccagcaccagggcctgtggaaaagccacccagagaacccgcaaggcagctacggaatcctccaaccaccattggaatgatgactctgaaagcagctttcagaactctgtctggtgcacaggattctgaggcagcctttgcaaaactggaccagaaggatctggttcttcctactcaagctctcccagcatcaccagccctcaaaaacaagagacccagaaaagatgaaaacgaaagttcagccccggctgacggtgagggtggctcggaactgcagcccaagaacaagcgcatgacaataagcagattggtcttggaggaggacagccagagtactgagcccagcgcaggcctcaactcctcccaggaggccgcttcagcgccaccatccaagcccaccgttctcaaccaacccctccctggagagaagaatcccaaagtacccaaaggcaagtggaacagctctaatggggttgaagaaaaggagacttgggtggaagaggatgaactgtttcaagttcaggcagcaccagatgaagacagtacaaccaatataacaaaaaagcagaagtggactgtagaagaaagcgagtgggtcaaggctggagtgcagaaatatggggaaggaaactgggctgccatttctaaaaattacccatttgttaaccgaacagctgtgatgattaaggatcgctggcggaccatgaaaagacttggcatgaacacgcgtacgcggccgctcgagcagaaactcatctcagaagaggatctggcagcaaatgatatcctggattacaaggatgacgacgataaggttggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacgctagcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcggggggggggggggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 2 - DNA, 5426 bp, synthetic construct
  misc_feature 1..5426: note = Plasmid Sequence of IL1R1 guideRNA
  misc_feature 318..339: note = Guide RNA Sequence
  misc_feature 1750..2460: note = mCherry Coding Sequence
  source 1..5426: mol_type = other DNA
ggccttttgctcacatgcgccattctcgagcatgcgccattctcgagcatgctcgagaatggcgcatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgccaatgggtggagtcttgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacctattcgccttaaggtctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgct

SEQ ID NO: 3 - DNA, 21 bp, synthetic construct
  misc_feature 1..21: note = sgRNA Sequence
  source 1..21: mol_type = other DNA
gctgccaatgggtggagtctt

SEQ ID NO: 4 - protein, 1910 aa, synthetic construct
  REGION 1..1910: note = Functional Protein Sequence of dCas9_TRF2_K293R
  REGION 1380..1879: note = TRF2 293 Arginine
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSMAGGGGSSDGSGRAAGRRASRSSGRARRGRHEPGLGGPAERGAGEARLEEAVNRWVLKFYFHEALRAFRGSRYGDFRQIRDIMQALLVRPLGKEHTVSRLLRVMQCLSRIEEGENLDCSFDMEAELTPLESAINVLEMIKTEFTLTEAVVESSRKLVKEAAVIICIKNKEFEKASKILKKHMSKDPTTQKLRNDLLNIIREKNLAHPVIQNFSYETFQQKMLRFLESHLDDAEPYLLTMAKKALKSESAASSTGKEDKQPAPGPVEKPPREPARQLRNPPTTIGMMTLKAAFRTLSGAQDSEAAFAKLDQKDLVLPTQALPASPALKNKRPRKDENESSAPADGEGGSELQPKNKRMTISRLVLEEDSQSTEPSAGLNSSQEAASAPPSKPTVLNQPLPGEKNPKVPKGKWNSSNGVEEKETWVEEDELFQVQAAPDEDSTTNITKKQKWTVEESEWVKAGVQKYGEGNWAAISKNYPFVNRTAVMIKDRWRTMKRLGMNTRTRPLEQKLISEEDLAANDILDYKDDDDKV

SEQ ID NO: 5 - DNA, 35 bp, synthetic construct
  misc_feature 1..35: note = Cloning Primer TRF2_BamHI
  misc_feature 4..9: note = BamHI Recognition Sequence
  source 1..35: mol_type = other DNA
aatggatccatggcgggaggaggcgggagtagcga

SEQ ID NO: 6 - DNA, 53 bp, synthetic construct
  misc_feature 1..53: note = Cloning Primer TRF2_NheI
  misc_feature 4..9: note = NheI Recognition Sequence
  source 1..53: mol_type = other DNA
aatgctagcaaccttatcgtcgtcatccttgtaatccaggatatcatttgctg

SEQ ID NO: 7 - DNA, 29 bp, synthetic construct
  misc_feature 1..29: note = Cloning Primer mCherry_AgeI
  misc_feature 5..10: note = AgeI Recognition Sequence
  source 1..29: mol_type = other DNA
tataaccggtcgccaccatggtgagcaag

SEQ ID NO: 8 - DNA, 37 bp, synthetic construct
  misc_feature 1..37: note = Cloning Primer mCherry_EcoRI
  misc_feature 5..10: note = EcoRI Recognition Sequence
  source 1..37: mol_type = other DNA
tatagaattcttacttgtacagctcgtccatgccgcc